(19) United States
(12) Patent Application Publication
(10) Pub. No.: US 2026/0159578 A1
(43) Pub. Date: Jun. 11, 2026

(54) TARGETS FOR REDUCING DEMENTIA BURDEN BY TARGETING BRAIN IMAGING-BASED ENDOPHENOTYPES OF DEMENTIA

(71) Applicant: Board of Regents, The University of Texas System, Austin, TX (US)

(72) Inventor: Muralidharan Sargurupremraj, San Antonio, TX (US)

(21) Appl. No.: 19/419,691

(22) Filed: Dec. 15, 2025

Related U.S. Application Data
(60) Provisional application No. 63/728,303, filed on Dec. 5, 2024.

(51) Int. Cl. C07K 16/18; A61K 45/06; C07K 16/28; C12Q 1/6883

(52) U.S. Cl. CPC C07K 16/18; A61K 45/06; C07K 16/28; C12Q 1/6883; C12Q 2600/136; C12Q 2600/156

(57) ABSTRACT

Methods provide a tractable roadmap for drug-target prioritization within the druggable genome by triangulating evidence from population genomic, transcriptomic and proteomic data. Multiple lines of evidence suggest certain drug targets to have a causative role in WMH burden and AD risk. This goes beyond biomarker functions, emphasizing drug-repurposing opportunities and supporting rationale for clinical trials. Additionally, the shared gene function among prioritized targets underscores the importance of post-translational modification in the AD disease process. Lastly, from a genetic epidemiology standpoint, our study provides novel insights into the connection between vascular brain injury, the coagulation cascade, and AD risk, including the possibility of specific coagulation components with potential causal roles.

### PRIORITY PARAGRAPH

This application claims benefit of U.S. Provisional Application No. 63/728,303 filed Dec. 5, 2024, which is incorporated herein by reference in its entirety.

### STATEMENT REGARDING FEDERALLY FUNDED RESEARCH

None.

### FIELD

Embodiments of the invention relate to the field of medicine, specifically the treatment of neurodegenerative diseases.

### BACKGROUND

Observational studies have revealed that most individuals who are cognitively healthy tend to remain so even when they have the characteristic AD pathology. This suggests that multiple other biological pathways may be involved in the Alzheimer's disease process. During middle age, environmental variables interacting with genetic factors could lead to the disruption of crucial molecular pathways such as those that maintain the vascular system. Consequently, these altered mechanisms preceding the neuropathological insults could further potentiate the neurodegenerative processes at an older age, emphasizing their potential as a better treatment target.

There is a need for additional therapies and methods for identifying therapies to ameliorate dementia associated with neurodegenerative disease such as Alzheimer's disease.

### SUMMARY

Cerebrovascular disease (CeVD) is a major player in the pathophysiology of dementia. Using the Mendelian randomization (MR) framework, a putative causal relation of white matter hyperintensity burden (WMH), a brain imaging marker of CeVD, has been shown to be associated with an increased risk of Alzheimer's disease (AD). As a natural extension, using drug-repurposing strategies, the inventor aimed to explore the causal relation of the targets of clinically approved drugs with WMH burden in order to reduce dementia (AD) burden. Alzheimer's disease is a neurodegenerative disorder where pathological processes can begin up to 30 years before clinical symptoms appear. The majority of individuals diagnosed with Alzheimer's disease also exhibit vascular pathologies during post-mortem examinations. Therefore, by targeting the vascular burden prevalent in middle-aged individuals, it may be possible to manage, prevent, or mitigate dementia (AD) burden in the future.

The present invention provides methods for identifying and repurposing drugs for the treatment or prevention of neurodegenerative diseases, particularly dementia and Alzheimer's disease (AD), by targeting brain imaging-based endophenotypes such as white matter hyperintensity (WMH) burden and related vascular pathways.

In one aspect, the invention provides a method for identifying a drug for the treatment of a neurodegenerative disease comprising selecting genetic variants as proxies for effects of a drug target, assessing subjects having a neurodegenerative condition relative to the selected genetic variants, identifying a drug target having a causal relationship with the neurodegenerative disease, and identifying a drug that modulates the drug target as a therapeutic for the neurodegenerative disease. In certain embodiments, the neurodegenerative disease is Alzheimer's disease or dementia, and the genetic variants include single-nucleotide polymorphisms (SNPs).

In certain embodiments, the drug target is selected from CALCRL, MAP3K11, NMT1, EPHB4, GALK1, PKD2, and combinations thereof. In further embodiments, the drug is an inhibitor of CALCRL (CGRPr), an inhibitor of MAP3K11 (MLK3), an inhibitor or modulator of NMT1, or an anticoagulant.

In another aspect, the invention provides methods of treating or preventing dementia in a subject comprising administering to the subject an effective amount of a CGRPr antagonist. Exemplary CGRPr antagonists include small-molecule gepants (e.g., rimegepant, ubrogepant, atogepant, zavegepant) and monoclonal antibodies targeting CGRP or CGRPr (e.g., erenumab, fremanezumab, galcanezumab, eptinezumab).

In yet another aspect, the invention provides methods of treating or preventing dementia in a subject comprising administering to the subject an effective amount of an inhibitor of mixed-lineage kinase 3 (MLK3).

In a further aspect, the invention provides methods of treating or preventing dementia in a subject comprising administering to the subject an effective amount of an anticoagulant or an inhibitor of the coagulation cascade.

In certain embodiments of the treatment methods, the subject has elevated white matter hyperintensity (WMH) burden as determined by brain imaging, or is at risk of developing dementia due to vascular risk factors.

The invention further provides pharmaceutical compositions comprising a CGRPr antagonist, an MLK3 inhibitor, an NMT1 modulator, or an anticoagulant, together with a pharmaceutically acceptable carrier, for use in treating or preventing dementia.

Additionally, the invention provides the use of a CGRPr antagonist, an MLK3 inhibitor, an NMT1 modulator, or an anticoagulant in the manufacture of a medicament for treating or preventing dementia.

The invention further provides pharmaceutical compositions comprising a drug identified by the methods herein (e.g., a CGRPr antagonist) and a pharmaceutically acceptable carrier. Also provided are kits comprising such compositions and instructions for use in treating or preventing dementia.

In some embodiments, the method further comprises measuring white matter hyperintensity (WMH) burden in the subject (e.g., via MRI) and selecting subjects with elevated WMH for treatment.

Other embodiments of the invention are discussed throughout this application. Any embodiment discussed with respect to one aspect of the invention applies to other aspects of the invention as well and vice versa. Each embodiment described herein is understood to be applicable to all aspects of the invention. It is contemplated that any embodiment discussed herein can be implemented with respect to any method, composition, or kit of the invention, and vice versa.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

As used herein, the following terms have the meanings set forth below unless clearly indicated otherwise.

Administering or Administration—The terms “administering” or “administration” refer to the delivery of a therapeutic agent, such as a drug or pharmaceutical composition, to a subject by any suitable route, including but not limited to oral, intravenous, intramuscular, subcutaneous, intrathecal, intraperitoneal, or topical administration.

Alzheimer's disease (AD)—A progressive neurodegenerative disorder characterized by cognitive decline, memory loss, and the accumulation of amyloid-beta plaques and neurofibrillary tangles in the brain. AD is the most common cause of dementia in older adults.

Cerebrovascular disease (CeVD)—A group of conditions affecting blood flow and blood vessels in the brain, including cerebral small vessel disease (cSVD), which contributes to vascular pathology and is implicated in the pathophysiology of dementia, including Alzheimer's disease.

Druggable genome—The subset of genes or gene products (such as proteins) that are considered suitable targets for therapeutic drug development, typically because they play a role in disease pathways or physiological processes that can be modulated by drugs.

Drug repurposing—The process of identifying new therapeutic uses for existing, clinically approved drugs or compounds in clinical development that are outside the scope of their original medical indications. Instead of developing new drugs from scratch, researchers investigate existing drugs to find potential applications in treating different diseases or conditions. This approach can significantly shorten the drug development timeline and reduce costs, as the safety profiles and pharmacokinetics of these drugs are already well-established through previous research and clinical trials.

Expression quantitative trait locus (eQTL)—A genetic locus containing one or more genetic variants (e.g., SNPs) that are associated with variation in gene expression levels.

Mendelian randomization (MR)—A genetic epidemiological method that uses genetic variants as instrumental variables to infer causal relationships between an exposure (e.g., gene expression or protein levels) and an outcome (e.g., disease risk), thereby reducing confounding and reverse causation biases common in observational studies.

Polygenic score (PGS)—A numerical score derived from the sum of an individual's risk alleles, weighted by their effect sizes on a trait, used to estimate genetic predisposition to that trait.

Protein quantitative trait locus (pQTL)—A genetic locus containing one or more genetic variants (e.g., SNPs) that are associated with variation in protein abundance levels.

Putative causal target (PCT)—A gene or gene product identified through genetic and statistical analyses (e.g., Mendelian randomization and fine-mapping) as having a likely causal relationship with a trait or disease outcome, such as white matter hyperintensity burden or Alzheimer's disease risk.

Subject-A mammal, preferably a human, including individuals having, suspected of having, or at risk of developing a neurodegenerative disease or dementia. The term encompasses patients diagnosed with the condition as well as those in preclinical or prodromal stages.

Therapeutically effective amount—An amount of a drug or pharmaceutical composition sufficient to achieve a desired biological or therapeutic result, such as amelioration, prevention, or delay of symptoms associated with dementia or a neurodegenerative disease.

White matter hyperintensity (WMH) burden-A brain imaging marker, typically observed on magnetic resonance imaging (MRI), reflecting areas of increased signal intensity in white matter presumed to be of vascular origin. WMH is a key feature of covert cerebral small vessel disease (cSVD) and is associated with cognitive decline and increased risk of dementia, including Alzheimer's disease.

Other terms not specifically defined herein shall be understood to have their ordinary meaning as used in the fields of genetics, neurology, pharmacology, and medicine.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. One example of a study workflow described below. PCT, Putative causal targets; WMH, White matter hyperintensity burden; AD, Alzheimer's disease; QTL, quantitative trait loci; eQTL, expression QTL; pQTL, protein QTL; MR, Mendelian randomization; PGS, polygenic score; j=SNPs mapping to the gene representing a pathway, βj=Effect allele of j SNPs, i=i^{th} individual, PGS [symbol] =Pathway-specific PGS.

FIG. 2. Causal effect estimation of prioritized PCTs with WMH and related traits in disease-related tissue types. Only showing PCT-trait pairs with significant MR association (P<0.05) for WMH, corresponding causal effect estimates with significant MR association for the other traits are annotated. Blue: eQTL-based estimates. Green: pQTL-based estimates. CAD: Coronary artery disease.

FIG. 3. Targetable pathways enriched for WMH and their polygenic score association with AD status. Color-coded denoting the representative pathway. Asterix (*) denote the association of the top PGS bin vs the reference (·).

FIG. 4. Targetable pathways enriched for WMH and the WMH-specific and WMH-SBP polygenic score association with AD status. Color-coded denoting the representative pathway. Asterix (*) denote the association of the top PGS bin vs the reference (·)

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

### I. Neurodegenerative Diseases

Alzheimer's disease (AD), the most common form of dementia, is a progressive and age-related neurodegenerative disease. As global life expectancy continues to increase, dementia prevalence is expected to reach 75 million by 2030,¹ making prevention or delaying strategies a top public health priority. While aging is a prominent risk factor, most cognitively unimpaired individuals with the presence of major hallmarks of AD pathology remain cognitively stable over time,² suggesting a possible additive or synergistic effect from other pathological processes. Indeed, a growing body of research implicates vascular risk factors and cerebrovascular disease in AD pathogenesis,³⁻⁵ possibly involving dysfunction of the neurovascular unit that precedes the neurodegenerative process.^{6,7} Recently, Mendelian randomization (MR) studies discussed a putative causal effect of vascular risk factors and brain imaging markers of covert cerebral small vessel disease (cSVD) with increased risk for dementia, including Alzheimer's type.^{8,9} By using genetic variations as an instrumental (proxy) variable for exposure and leveraging their natural randomization at conception, MR better mitigates the risks of confounding and reverse causation, as seen in observational studies.¹⁰ Targeting vascular risk factors such as hypertension, though, holds promise in preventing dementia. Meta-analyses of clinical trials examining the effect of antihypertensive medications reveal a reduced risk of secondary outcomes (cognitive impairment) rather than dementia in isolation. Moreover, the distinct u-shaped association of blood pressure with dementia in the older age group poses a major challenge in determining the optimal blood pressure range for dementia prevention and management.^{11,12} This underscores the requirement for a more stable marker to target that accurately reflects the progression of the disease.

White matter hyperintensities (WMH) of presumed vascular origin—the most common magnetic resonance imaging (MRI) feature of covert cSVD, exhibit a dose-dependent relationship with both cognitive decline and dementia;^{13,14} and are considered to be the main pathological substrate.¹⁵ They are known to interact with neurodegenerative lesions, which can potentiate or accelerate the clinical expression of AD. Recently, using multivariable MR adjusting for the possible confounding factors, a direct causal link of WMH with AD risk was discussed.¹⁶ Altogether, highlighting WMH as an optimal treatment target for reducing the burden of dementia, including AD.

Indeed, with the availability of single-nucleotide polymorphism (SNP) association data for clinical and cellular phenotypes (e.g. gene expression, protein levels) from genome-wide association studies (GWASs) and quantitative trait loci (QTLs) analysis, respectively, it is now increasingly feasible to apply MR principles to explore the effects of existing drugs with traits for which the drug was not originally designed.¹⁷ In general, drug-target MR assigns SNPs in genes encoding protein drug targets as instrumental variables, proxying the drug effect based on the SNP effects from the corresponding intermediate traits (e.g., lowering blood pressure, lipid levels, etc.).¹⁸ Nevertheless, GWASs for such intermediate exposures and clinical outcomes^{9,19-21} emphasize the underlying polygenic architecture involving multiple pathways and widespread pleiotropy.²² Failure to account for these pleiotropic effects could introduce bias in the causal effect estimates. Moreover, the aggregate effect of drug targets within a drug class may not fully capture all drug compound and target interactions, posing challenges in comparison with clinical trials. 23

### II. Cellular Phenotypes in Triangulating Putative Drug Targets for Alzheimer's Disease Endophenotypes.

A hypothesis-free approach was adopted by systematically studying the target-specific causal effects of the druggable genome,²⁴ in relation to WMH and related traits and AD. The term “druggable genome” refers to the subset of genes or gene products (such as proteins) that are considered suitable targets for therapeutic drug development. These targets typically play a role in disease pathways or physiological processes that can be modulated by drugs to treat or prevent diseases. By using cellular phenotypes of drug-targets as the exposures, from multiple disease-relevant tissue sources (blood, brain, cerebrospinal fluid-CSF) including non-publicly available datasets (Framingham Heart Study, FHS; Fundació ACE), we better proxy their biological effects triangulating any putative causal evidence to prioritize relevant therapeutic targets. Specifically, in a probabilistic statistical framework we studied the target-specific effect of the drug-targets,²⁵ thereby addressing the off-target effects due to “molecular pleiotropy”—i.e., a single eQTL regulating the expression of multiple genes or drug-targets. In an earlier study, we found that nearly one-third of the risk variants predisposing to WMH are not mediated by conventional risk factors, suggesting alternative pathophysiological mechanisms.⁹ Thus, as a second arm of our objective, we extend to test pathways enriched in WMH for potential modulation by pharmacological compounds. Additionally, we further study the association of WMH-enriched druggable pathways with AD status using a polygenic score approach.

### A. Methods

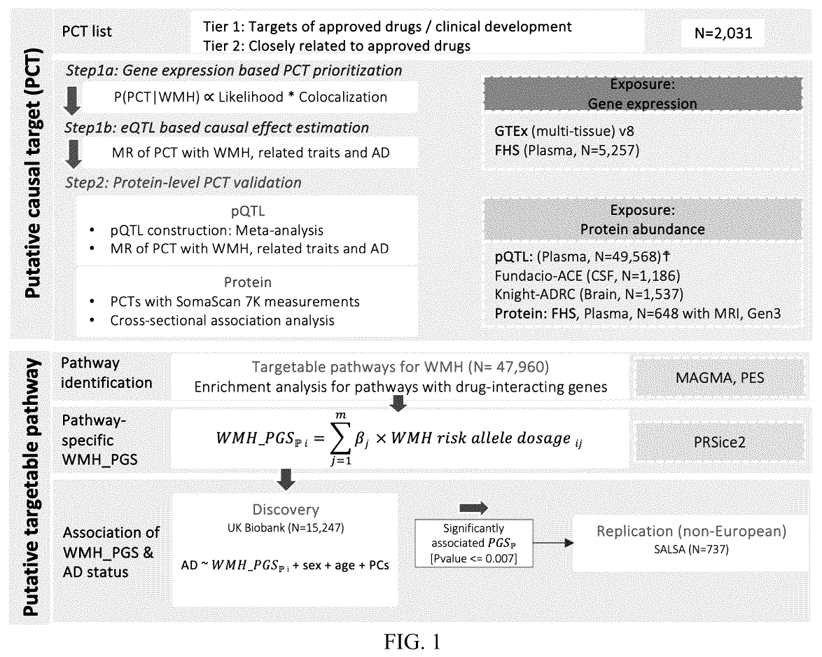

The outline of a study design is summarized in FIG. 1.

Druggable genome: Using the druggable genome described in detail elsewhere,²⁴ the drug targets were identified. Briefly, only autosomal and protein-coding genes (N=2,031) that are annotated to be the targets of approved drugs or drugs in clinical development (Tier 1) and drug-like compounds that are closely related to the approved drug targets (Tier 2) were considered for the drug-target causal analyses scheme.

Exposures and outcome variables: For the exposures, we considered genetic instruments predicting the cellular phenotype levels (gene expression-eQTLs, protein levels-pQTLs) of the druggable genome. In step 1, gene expression data from the FHS and the GTEx project (v.8) were considered.²⁶ We validated the putative causal signals identified in step 1 at the level of protein abundance from the FHS and pQTL information from the Spanish Fundacio-ACE cohort (N=1186) and publicly available datasets. 27—As the primary outcome, GWAS summary statistics were used for WMH (N=48,454) and additionally considered other WMH-related traits from the latest and largest GWASs (Table 4). A second objective was to identify the putative causal pathways with druggable potential, individual-level genotype and dementia/AD ascertainment based on the International Classification of Diseases (ICD9 and ICD10) coding system from the UK biobank study was used as the outcome. As a cross-ancestry replication effort, the dementia/AD ascertainment and genotype information from the Sacramento Area Latino Study on Aging (SALSA) was also used, an epidemiological study of health and cognition in Hispanics aged 60 and over (N=1,499).

TABLE 4
GWAS summary statistics used for the PCT prioritization
and causal effect estimation

| Trait | Population | Sample size | PMID |
|---|---|---|---|
| WMH | European | 49,864 | 33293549 |
| PWMH | European | 26,654 | 32517579 |
| DMWH | European | 26,654 | 32517579 |
| AD | European | 94,437; Ncases = 21,982 | 30820047 |
| AD-parental | European | 314,278; Ncases = 42,034 | 29777097 |
| AD-meta | European | 788,989; Ncases = 111,326 | 35379992 |
| All stroke | European | 1,308,460; Ncases = 73,652 | 36180795 |
| Migraine | European | 202,140; Ncases = 29,209 | 27322543 |
| CAD | European | 184,305; Ncases = 60,801 | 26343387 |

WMH: White matter hyperintensity burden;
PWMH: Periventricular WMH;
DWMH: deep WMH;
CAD: Coronary artery disease;
AD: Alzheimer's disease

Putative causal drug-targets (PCT): eQTL fine-mapping and TWAS-We first conducted probabilistic fine-mapping of the eQTL variants for each gene coding the drug targets using Deterministic Approximation of Posteriors (DAP-G)³⁰ in the FHS dataset. Briefly, DAP-G uses individual-level genotype, gene expression data and covariates to compute (i) a gene-level posterior inclusion probability (PIP) for harboring at least one causal variant and (ii) a variant-level PIP that is causal for the gene expression. Only common variants falling within 500 Kb of the gene boundary were considered, and the analysis model included age at blood collection, sex and other cohort-specific variables as covariates. As an independent dataset, pre-computed fine-mapping results from the GTEx multi-tissue data (v8) were additionally considered. Second, the fine-mapping results from DAP-G and the SNP-WMH effect estimates from the largest WMH GWAS⁹ were analyzed to estimate (i) the association statistic (Z-score) of the fine-mapped eQTLs with WMH in a TWAS framework,³¹ and (ii) the gene-level colocalization probability for shared causal variants between gene expression and WMH levels.³²

PCT prioritization-TWAS and colocalization methods exhibit low specificity and sensitivity respectively,³³ for detecting causal signals, leading to issues such as pleiotropy. This is primarily attributed to linkage disequilibrium (LD) structure, as any inferred causal signal between gene expression and the complex trait by these methods could be biased by the LD between eQTL and trait-associated SNPs. To address these challenges, we utilized INTACT, a probabilistic integration method known for its robustness in handling LD-related issues.²⁵ Briefly, INTACT combines the TWAS association statistic and gene-level colocalization probability using a shrinkage parameter. This parameter ensures that genes that lack colocalization evidence due to LD are excluded from the analysis, fulfilling one of the tenets of IV assumptions (exclusion restriction)—no pleiotropic effect from the target gene to a complex trait of interest. An INTACT-posterior probability greater than 0.9 was considered indicative of the presence of a putative causal effect of gene expression on the complex trait. For the prioritized targets, first, we tested the interaction of PCTs with pharmacological compounds using the Drug-Gene Interaction Database (DGIdb, URL www.dgidb.org), providing information on drug-gene interactions from various sources. Second, we tested the association of the prioritized targets with other complex traits using Genebass, a browser (URL genebass.org) framework for gene-based phenome-wide association study (PheWAS).

Causal effect estimation-Finally, in a Mendelian randomization setting,³⁴ the putative causal effect of the prioritized PCTs on the primary (WMH) and other related traits (Table 4), was estimated. For constructing the genetic instruments, only fine-mapped eQTLs from the DAP-G analysis in step 1 and satisfying the instrumental variable definition,^{35,36} individually in FHS³⁷ and GTEx dataset³⁸ were considered. The genetic instruments for each PCTs were further pruned by including only LD-independent SNPs (r2<0.1, based on 1000 genomes European panel) (Table 5). Effect alleles of the genetic instruments were defined as the allele associated with an increase in the corresponding gene expression values.

TABLE 5
PCT Instrumental variables (IVs) satisfying fine-mapping criteria and individual eQTL study criteria for the MR analyses

| | | | Allele | Allele | | | grouping |
|---|---|---|---|---|---|---|---|
| EnsemblID: Tissue_type | PCT-HGNC | MarkerName | Effect | Other | Beta | StdErr | Tissue |
| ENSG00000143437: Artery_Tibial | ARNT | rs12758227 | C | A | 0.218 | 0.021 | Vascular |
| ENSG00000064989: Artery_Aorta | CALCRL | rs62172372 | G | A | 0.527 | 0.049 | Vascular |
| ENSG00000064989: Artery_Coronary | CALCRL | rs8176526 | T | C | 0.475 | 0.073 | Vascular |
| ENSG00000064989: Artery_Tibial | CALCRL | rs62172372 | G | A | 0.789 | 0.037 | Vascular |
| ENSG00000064989: Brain_Cerebellum | CALCRL | rs698588 | G | A | 0.253 | 0.046 | Neurological |
| ENSG00000064989: FHS_blood | CALCRL | rs12987470 | A | T | 0.030 | 0.002 | Vascular |
| ENSG00000064989: Nerve_Tibial | CALCRL | rs62172421 | C | T | 0.298 | 0.050 | Neurological |
| ENSG00000196411: Artery_Aorta | EPHB4 | rs2250818 | A | G | 0.131 | 0.030 | Vascular |
| ENSG00000196411: Heart_Left_Ventricle | EPHB4 | rs7384255 | T | C | 0.149 | 0.031 | Vascular |
| ENSG00000173327: Artery_Aorta | MAP3K11 | rs2004649 | A | G | 0.371 | 0.031 | Vascular |
| ENSG00000173327: Artery_Coronary | MAP3K11 | rs2004649 | A | G | 0.319 | 0.040 | Vascular |
| ENSG00000173327: Brain_Caudate_basal_ganglia | MAP3K11 | rs1144789 | A | G | 0.148 | 0.035 | Neurological |
| ENSG00000173327: Brain_Cerebellar_Hemisphere | MAP3K11 | rs4930319 | C | G | 0.208 | 0.050 | Neurological |
| ENSG00000173327: Brain_Cerebellum | MAP3K11 | rs1144789 | A | G | 0.281 | 0.041 | Neurological |
| ENSG00000173327: Brain_Cortex | MAP3K11 | rs1144789 | A | G | 0.189 | 0.041 | Neurological |
| ENSG00000173327: Brain_Frontal_Cortex_BA9 | MAP3K11 | rs61744384 | A | T | 0.182 | 0.039 | Neurological |
| ENSG00000136448: Artery_Coronary | NMT1 | rs12936234 | T | C | 0.212 | 0.033 | Vascular |
| ENSG00000136448: Brain_Caudate_basal_ganglia | NMT1 | rs55897749 | T | C | 0.348 | 0.048 | Neurological |
| ENSG00000136448: Brain_Cerebellar_Hemisphere | NMT1 | rs12936234 | T | C | 0.245 | 0.052 | Neurological |
| ENSG00000136448: Brain_Cerebellum | NMT1 | rs4793173 | A | C | 0.305 | 0.050 | Neurological |
| ENSG00000136448: Brain_Cortex | NMT1 | rs4793179 | C | T | 0.136 | 0.030 | Neurological |
| ENSG00000136448: Brain_Frontal_Cortex_BA9 | NMT1 | rs2239919 | A | G | 0.158 | 0.031 | Neurological |
| ENSG00000136448: Brain_Putamen_basal_ganglia | NMT1 | rs7213273 | G | A | 0.348 | 0.055 | Neurological |
| ENSG00000136448: Brain_Spinal_cord_cervical_c-1 | NMT1 | rs4793173 | A | C | 0.420 | 0.069 | Neurological |
| ENSG00000136448: Nerve_Tibial | NMT1 | rs6416904 | A | G | 0.256 | 0.020 | Neurological |
| ENSG00000118762: Brain_Cerebellar_Hemisphere | PKD2 | rs2728104 | C | T | 0.263 | 0.059 | Neurological |
| ENSG00000065243: FHS_blood | PKN2 | rs786907 | C | T | 0.007 | 0.001 | Vascular |
| ENSG00000130304: Brain_Cerebellum | SLC27A1 | rs34761413 | C | G | 0.437 | 0.043 | Neurological |
| ENSG00000065613: Brain_Cerebellum | SLK | rs10883936 | T | C | 0.274 | 0.059 | Neurological |

MR: Mendelian randomization;
PCT-HGNC: Putative causal target - Human genome nomenclature symbol

Protein-level PCT validation-Using pQTLs predicting protein abundance, we further validated the concordance in the causal effect direction of PCTs prioritized in step 1. Employing a meta-analysis strategy, we first constructed pQTL genetic instruments for each PCT from publicly available pQTL datasets (plasma, cerebrospinal fluid-CSF)^{27-29,39} and CSF protein levels-SNP association in the Spanish Fundacio-ACE cohort (N=1186). LD-independent SNPs (r2<0.1) within 1 Mb of the PCT transcription start site served as genetic instruments. A larger p-value threshold (P<0.001) ensured adequate genetic instruments per PCT, while maintaining the causal estimation accuracy.^{40,41} Causal effect estimates with the different outcomes were obtained using the standard IVW method and multiple pleiotropy-robust MR methods (MR-RAPS, MR-Egger, weighted median). Consistent estimates in the IVW and two or more pleiotropy-robust methods were considered indicative of the presence of causal association.⁴² Using colocalization analysis,⁴³ additional pleiotropic effects due to LD were ruled out. A posterior probability (PP4)≥75% indicated evidence for a shared causal variant between protein abundance and trait levels/risk. Finally, PCTs protein-level association with hippocampal volume was tested in the FHS (N=956).

Putative targetable pathways: Pathway identification—To identify pathways enriched for WMH that can be modulated by pharmacological compounds, we employed the pharmagenic enrichment score (PES) pipeline.⁴⁴ Using MAGMA and p-value thresholding (P_{T}), PES generates gene-based association statistics from the WMH GWAS, based on the association strength at different polygenic levels (all SNPs, P<0.5, P<0.05, and P<0.005). Only variants proximal (5 Kb upstream; 1.5 Kb downstream) to a protein-coding gene were considered. Second, at each P_{T} bin, using pre-defined druggable gene sets (MSigDB) competitive association test was conducted, testing the null hypothesis of genes in the set being no more strongly associated than all other genes. Given the polygenic nature of WMH,¹⁹ and as suggested elsewhere,⁴⁴ a significance threshold of p<001 was applied to maximize the identification of putative pathways.

Pathway association with AD—In a polygenic score scheme, we assessed whether the druggable pathways enriched for WMH were also associated with AD status. First using PRSice^{2,45} WMH risk SNPs mapping to genes constituting the clinically actionable pathways were identified. Second, in the discovery dataset (UK Biobank, Europeans) the pathway-specific WMH-PGS were generated by weighting the number of WMH-risk ‘effect’ alleles times the effect estimates from the WMH GWAS. Third, using the strata analysis feature in PRSice2, we aggregated the AD cases and controls in the UK Biobank into five strata based on the increasing WMH-PGS for a given candidate pathway. A logistic regression adjusting for age, sex and principal components was conducted to estimate the relative risk of each stratum vs the reference set. For pathways showing a significant association of the WMH-PGS with AD status, we additionally replicated the findings in a non-European cohort (SALSA, Hispanics), testing pathway-specific WMH-PGS with AD status. We took care to ensure there was no sample overlap between the individuals included in the WMH GWAS and in our test datasets.

### B. Results:

Drug targets and WMH: prioritization, causal effects, and implications for AD. Leveraging fine-mapped eQTLs for each gene coding the drug targets; our prioritization method revealed an average non-zero gene-level colocalization probability for 1,634 drug targets across 18 tissue types, indicating likely shared variants between gene expression and WMH levels (Table 6). Further, we integrated the gene-level colocalization probability with TWAS association strength and accounted for potential pleiotropic effects through INTACT. This effectively prioritized 11 PCTs across 12 tissue types with a high posterior probability (>0.90) of a causal relationship between gene expression levels and WMH burden (Table 7). The majority (n=8) represent targets of clinically-informed drugs (Tier 1), and three (NMT1, GALK1, PKD2) PCTs are targets of pharmacological compounds that are in developmental stages. Testing the interaction of the prioritized PCTs with pharmacological compounds identified inhibitory effects as the nature of the drug-gene interactions (Table 8). We, therefore, sought to study the loss of function of the genes coding the prioritized PCTs in a PheWAS setting. Specifically, focusing on the aggregate effect of loss-of-function variants in a gene-based burden test scheme showed prominent associations with digestive disorders (Table 9).

TABLE 6
PCTs exhibiting non-zero colocalization probability
for shared variants between gene expression and WMH

| Tissue type | Study | N PCTs |
|---|---|---|
| Whole_blood | FHS | 1895 |
| Artery_Aorta | GTEx | 1628 |
| Artery_Coronary | GTEx | 1649 |
| Artery_Tibial | GTEx | 1591 |
| Brain_Amygdala | GTEx | 1639 |
| Brain_Anterior_cingulate_cortex_BA24 | GTEx | 1632 |
| Brain_Caudate_basal_ganglia | GTEx | 1649 |
| Brain_Cerebellar_Hemisphere | GTEx | 1636 |
| Brain_Cerebellum | GTEx | 1644 |
| Brain_Cortex | GTEx | 1646 |
| Brain_Hippocampus | GTEx | 1640 |
| Brain_Hypothalamus | GTEx | 1686 |
| Brain_Nucleus_accumbens_basal_ganglia | GTEx | 1639 |
| Brain_Putamen_basal_ganglia | GTEx | 1598 |
| Brain_Spinal_cord_cervical_c-1 | GTEx | 1654 |
| Brain_Substantia_nigra | GTEx | 1632 |
| Heart_Atrial_Appendage | GTEx | 1578 |
| Heart_Left_Ventricle | GTEx | 1512 |
| Whole_Blood | GTEx | 1503 |

GTEx: Genotype tissue expression project;
FHS: Framingham heart study

TABLE 7
PCTs with >90% colocalization probability for shared causal variants between gene expression and WMH

| | | | | INTACT | | | |
|---|---|---|---|---|---|---|---|
| | | | TWAS | colocalization | Tissue | Tissue | |
| EnsemblID | PCT-HGNC | Druggability | Z-Score | probability | type | grouping | Study |
| ENSG00000143437 | ARNT | Tier 1 | 4.549 | 0.994 | Artery_Tibial | Vascular | GTEx |
| ENSG00000064989 | CALCRL | Tier 1 | 6.213 | 1.000 | Whole_blood | Vascular | FHS |
| ENSG00000064989 | CALCRL | Tier 1 | −6.458 | 1.000 | Artery_Aorta | Vascular | GTEx |
| ENSG00000064989 | CALCRL | Tier 1 | −6.381 | 1.000 | Artery_Coronary | Vascular | GTEx |
| ENSG00000064989 | CALCRL | Tier 1 | −6.458 | 1.000 | Artery_Tibial | Vascular | GTEx |
| ENSG00000064989 | CALCRL | Tier 1 | 5.917 | 1.000 | Brain_Cerebellum | Neurological | GTEx |
| ENSG00000196411 | EPHB4 | Tier 1 | 4.088 | 0.952 | Artery_Aorta | Vascular | GTEx |
| ENSG00000196411 | EPHB4 | Tier 1 | 4.159 | 0.957 | Heart_Left_Ventricle | Vascular | GTEx |
| ENSG00000108479 | GALK1 | Tier 2 | 10.955 | 1.000 | Brain_Cerebellum | Neurological | GTEx |
| ENSG00000173327 | MAP3K11 | Tier 1 | 4.000 | 0.903 | Artery_Aorta | Vascular | GTEx |
| ENSG00000173327 | MAP3K11 | Tier 1 | 4.000 | 0.909 | Artery_Coronary | Vascular | GTEx |
| ENSG00000173327 | MAP3K11 | Tier 1 | 4.000 | 0.929 | Brain_Caudate_basal_ganglia | Neurological | GTEx |
| ENSG00000173327 | MAP3K11 | Tier 1 | 4.102 | 0.907 | Brain_Cerebellar_Hemisphere | Neurological | GTEx |
| ENSG00000173327 | MAP3K11 | Tier 1 | 4.000 | 0.927 | Brain_Cerebellum | Neurological | GTEX |
| ENSG00000173327 | MAP3K11 | Tier 1 | 4.000 | 0.933 | Brain_Cortex | Neurological | GTEx |
| ENSG00000173327 | MAP3K11 | Tier 1 | 4.000 | 0.909 | Brain_Putamen_basal_ganglia | Neurological | GTEx |
| ENSG00000136448 | NMT1 | Tier 2 | −7.421 | 1.000 | Artery_Coronary | Vascular | GTEx |
| ENSG00000136448 | NMT1 | Tier 2 | −8.610 | 1.000 | Brain_Caudate_basal_ganglia | Neurological | GTEx |
| ENSG00000136448 | NMT1 | Tier 2 | −7.421 | 1.000 | Brain_Cerebellar_Hemisphere | Neurological | GTEx |
| ENSG00000136448 | NMT1 | Tier 2 | −8.377 | 1.000 | Brain_Cerebellum | Neurological | GTEx |
| ENSG00000136448 | NMT1 | Tier 2 | −7.949 | 1.000 | Brain_Cortex | Neurological | GTEX |
| ENSG00000136448 | NMT1 | Tier 2 | −7.903 | 1.000 | Brain_Putamen_basal_ganglia | Neurological | GTEx |
| ENSG00000136448 | NMT1 | Tier 2 | −8.377 | 1.000 | Brain_Spinal_cord_cervical_c-1 | Neurological | GTEx |
| ENSG00000136448 | NMT1 | Tier 2 | −7.949 | 1.000 | Brain_Substantianigra | Neurological | GTEx |
| ENSG00000164867 | NOS3 | Tier 1 | −4.313 | 0.954 | Artery_Tibial | Vascular | GTEx |
| ENSG00000118762 | PKD2 | Tier 2 | −4.151 | 0.948 | Brain_Cerebellar_Hemisphere | Neurological | GTEx |
| ENSG00000065243 | PKN2 | Tier 1 | 4.474 | 0.985 | Whole_blood | Vascular | FHS |
| ENSG00000130304 | SLC27A1 | Tier 1 | −3.984 | 0.907 | Brain_Cerebellum | Neurological | GTEx |
| ENSG00000065613 | SLK | Tier 1 | 4.469 | 0.916 | Artery_Aorta | Vascular | GTEx |
| ENSG00000065613 | SLK | Tier 1 | 6.517 | 1.000 | Brain_Cerebellum | Neurological | GTEx |

TABLE 8
Prioritized PCTs and interaction with pharmacological compound

| Match | PCT- | | | score | | |
|---|---|---|---|---|---|---|
| type | HGNC | Drug name | type Interaction | Interaction | Source | PMID |
| Definite | CALCRL | DAVALINTIDE | Agonist | 1.156 | ChemblInteractions | NA |
| Definite | CALCRL | OLCEGEPANT | Antagonist | 6.939 | ChemblInteractions\|TTD | 28165287 |
| Definite | CALCRL | TELCAGEPANT | Antagonist | 6.939 | ChemblInteractions\|TTD | 20573757 |
| Definite | CALCRL | MK3207 | Antagonist | 3.469 | ChemblInteractions\|TTD | NA |
| Definite | CALCRL | RIMEGEPANT | Antagonist | 2.313 | ChemblInteractions\|TTD | 31291516\|31081399 |
| Definite | CALCRL | UBROGEPANT | Antagonist | 1.735 | ChemblInteractions | NA |
| Definite | CALCRL | ERENUMAB | antagonist\|antibody | 6.939 | ChemblInteractions\|TTD | NA |
| Definite | MAP3K11 | CEP-1347 | inhibitor | 19.660 | DTC\|ChemblInteractions | 24044867 |
| Definite | EPHB4 | TESEVATINIB | inhibitor | 10.532 | TALC\|MyCancerGenome\| | NA |
| | | | | | TdgClinicalTrial\| | |
| | | | | | ChemblInteractions\|TTD | |
| Definite | EPHB4 | JI-101 | inhibitor | 4.915 | ChemblInteractions | NA |
| Definite | EPHB4 | TG100-801 | inhibitor | 0.922 | ChemblInteractions | NA |
| Definite | EPHB4 | VANDETANIB | inhibitor | 0.273 | ChemblInteractions | NA |
| Definite | SLK | HESPERADIN | inhibitor | 0.049 | DTC | 19035792 |
| Definite | PKN2 | QUERCETIN | inhibitor | 0.036 | MyCancerGenome | NA |
| Definite | NOS3 | (−)-EPICATECHIN | NA | NA | DTC | 24794111 |
| Definite | NOS3 | CLOPIDOGREL | NA | NA | PharmGKB | 22890915 |
| Definite | NOS3 | CYCLOPHOSPHAMIDE | NA | NA | PharmGKB | 19671875\|25545243\| |
| | | | | | | 29938344 |
| Definite | NOS3 | DOXORUBICIN | NA | NA | PharmGKB | 19671875\|25545243\| |
| | | | | | | 29938344\|21048526 |
| Definite | NOS3 | ENALAPRIL | NA | NA | PharmGKB | 22706620 |
| Definite | NOS3 | EPIRUBICIN | NA | NA | PharmGKB | 19671875\|25545243 |
| Definite | NOS3 | FLUOROURACIL | NA | NA | PharmGKB | 19671875\|25545243\| |
| | | | | | | 29938344 |
| Definite | NOS3 | OXALIPLATIN | NA | NA | PharmGKB | 19671875\|25545243 |
| Definite | PKN2 | CHEMBL225519 | NA | NA | DTC | NA |
| Definite | PKN2 | CHEMBL541400 | NA | NA | DTC | NA |
| Definite | SLK | CHEMBL225519 | NA | NA | DTC | NA |
| Definite | SLK | CHEMBL541400 | NA | NA | DTC | NA |
| Definite | SLK | CHIR-99021 | NA | NA | DTC | NA |
| Definite | SLK | OSI-632 | NA | NA | DTC | NA |
| Definite | SLK | R-406 | NA | NA | DTC | NA |
| Definite | ARNT | FUROSPONGOLIDE | NA | 11.796 | DTC | 18989978 |
| Definite | ARNT | EPICATECHIN | NA | 5.898 | DTC | 15620252 |
| | | GALLATE | | | | |
| Definite | NOS3 | ARGININE | NA | 4.718 | PharmGKB | 16682803\|16690332\| |
| | | | | | | 16717106\|26385052\| |
| | | | | | | 16729278\|16720041 |
| Definite | PKD2 | ALLOPURINOL | NA | 4.068 | PharmGKB | 30924126 |
| Definite | CALCRL | CALCITONIN GENE- | NA | 3.469 | TdgClinicalTrial | NA |
| | | RELATED PEPTIDE | | | | |
| Definite | NOS3 | L-NAME | NA | 2.359 | TTD | NA |
| Definite | NOS3 | RONOPTERIN | NA | 2.359 | TTD | NA |
| Definite | CALCRL | FREMANEZUMAB | NA | 1.735 | TTD | NA |
| Definite | GALK1 | PYRANTEL PAMOATE | NA | 1.229 | DTC | NA |
| Definite | NOS3 | SAPROPTERIN | NA | 1.180 | TdgClinicalTrial\|TEND | NA |
| Definite | CALCRL | GALCANEZUMAB | NA | 1.156 | TTD | NA |
| Definite | NOS3 | SILDENAFIL | NA | 0.674 | PharmGKB | 22064666 |
| Definite | ARNT | HYDROCORTISONE | NA | 0.638 | NCI | 10048155 |
| Definite | GALK1 | TRICETIN | NA | 0.614 | DTC | NA |
| Definite | GALK1 | CHEMBL1498652 | NA | 0.492 | DTC | NA |
| Definite | GALK1 | SURAMIN | NA | 0.410 | DTC | NA |
| | | HEXASODIUM | | | | |
| Definite | NOS3 | SPIRONOLACTONE | NA | 0.278 | PharmGKB | 22543981 |
| Definite | GALK1 | CHEMBL601757 | NA | 0.205 | DTC | NA |
| Definite | GALK1 | 9,10- | NA | 0.189 | DTC | NA |
| | | PHENANTHRENEQUINONE | | | | |
| Definite | NOS3 | GEDATOLISIB | NA | 0.189 | DTC | 20166697 |
| Definite | GALK1 | CHEMBL578512 | NA | 0.169 | DTC | NA |
| Definite | GALK1 | AURINTRICARBOXYLIC | NA | 0.159 | DTC | NA |
| | | ACID | | | | |
| Definite | NOS3 | DIGOXIN | NA | 0.143 | PharmGKB | 22543981 |
| Definite | NOS3 | IBUPROFEN | NA | 0.135 | PharmGKB | 25502615 |
| Definite | NOS3 | SUNITINIB | NA | 0.124 | DTC | 21963305 |
| Definite | GALK1 | HEXACHLOROPHENE | NA | 0.123 | DTC | NA |
| Definite | PKN2 | LY-2090314 | NA | 0.108 | DTC | NA |
| Definite | GALK1 | GOSSYPOL | NA | 0.102 | DTC | NA |
| Definite | GALK1 | CHEMBL429095 | NA | 0.098 | DTC | NA |
| Definite | SLK | TAK-715 | NA | 0.082 | DTC | NA |
| Definite | SLK | LY-2090314 | NA | 0.076 | DTC | NA |
| Definite | SLK | SB-203580 | NA | 0.076 | DTC | NA |
| Definite | NOS3 | METHOTREXATE | NA | 0.072 | PharmGKB | 19671875\|25545243\| |
| | | | | | | 29938344 |
| Definite | NOS3 | DAUNORUBICIN | NA | 0.068 | PharmGKB | 24684492 |
| Definite | NOS3 | ASPIRIN | NA | 0.067 | PharmGKB | 22890915 |
| Definite | PKN2 | CHEMBL379975 | NA | 0.064 | DTC | NA |
| Definite | PKN2 | GSK-269962A | NA | 0.064 | DTC | NA |
| Definite | GALK1 | QUERCETIN | NA | 0.062 | DTC | NA |
| Definite | PKN2 | ALSTERPAULLONE | NA | 0.056 | DTC | NA |
| Definite | PKN2 | ERLOTINIB | NA | 0.056 | DTC | NA |
| Definite | PKN2 | SOTRASTAURIN | NA | 0.051 | DTC | NA |
| Definite | SLK | GW843682X | NA | 0.050 | DTC | NA |
| Definite | PKN2 | ENTRECTINIB | NA | 0.049 | DTC | NA |
| Definite | SLK | SNS-314 | NA | 0.047 | DTC | NA |
| Definite | SLK | ALISERTIB | NA | 0.045 | DTC | NA |
| Definite | SLK | GSK-269962A | NA | 0.045 | DTC | NA |
| Definite | PKN2 | GW441756X | NA | 0.043 | DTC | NA |
| Definite | SLK | CEDIRANIB | NA | 0.041 | DTC | NA |
| Definite | PKN2 | TOZASERTIB | NA | 0.040 | DTC | NA |
| Definite | PKN2 | DASATINIB | NA | 0.040 | DTC | NA |
| Definite | SLK | ERLOTINIB | NA | 0.039 | DTC | NA |
| Definite | SLK | SOTRASTAURIN | NA | 0.036 | DTC | NA |
| Definite | SLK | ENTRECTINIB | NA | 0.034 | DTC | NA |
| Definite | PKN2 | RG-1530 | NA | 0.033 | DTC | NA |
| Definite | SLK | GEFITINIB | NA | 0.032 | DTC | NA |
| Definite | PKN2 | DOVITINIB | NA | 0.032 | DTC | NA |
| Definite | SLK | GW441756X | NA | 0.030 | DTC | NA |
| Definite | PKN2 | CYC-116 | NA | 0.029 | DTC | NA |
| Definite | SLK | LINIFANIB | NA | 0.028 | DTC | NA |
| Definite | SLK | TOZASERTIB | NA | 0.028 | DTC | NA |
| Definite | SLK | DASATINIB | NA | 0.028 | DTC | NA |
| Definite | PKN2 | TAE-684 | NA | 0.028 | DTC | NA |
| Definite | PKN2 | PF-00562271 | NA | 0.027 | DTC | NA |
| Definite | PKN2 | ILORASERTIB | NA | 0.027 | DTC | NA |
| Definite | NOS3 | SORAFENIB | NA | 0.025 | PharmGKB | 27058899 |
| Definite | PKN2 | CENISERTIB | NA | 0.024 | DTC | NA |
| Definite | SLK | RG-1530 | NA | 0.023 | DTC | NA |
| Definite | SLK | DOVITINIB | NA | 0.022 | DTC | NA |
| Definite | SLK | SP-600125 | NA | 0.022 | DTC | NA |
| Definite | SLK | SORAFENIB | NA | 0.020 | DTC | NA |
| Definite | SLK | CYC-116 | NA | 0.020 | DTC | NA |
| Definite | SLK | TAE-684 | NA | 0.019 | DTC | NA |
| Definite | SLK | PF-00562271 | NA | 0.019 | DTC | NA |
| Definite | SLK | ILORASERTIB | NA | 0.019 | DTC | NA |

TABLE 9
Phenome-wide scan of PCTs targeted by inhibitors and antagonists

| PCT- | | | | N | N | | | P-Value |
|---|---|---|---|---|---|---|---|---|
| HGNC | Phenotype | Trait type | Category | cases | controls | Burden test | Beta | (SKAT-O) |
| PKN2 | Height | continuous | Anthropometry | 393982 | NA | Loss-of-function | 0.018 | 4.43E−05 |
| PKN2 | Height | continuous | Anthropometry | 393982 | NA | Loss-of-function | 0.018 | 7.02E−05 |
| EPHB4 | Cystatin C | continuous | Blood | 376784 | NA | Loss-of-function | 0.021 | 1.95E−05 |
| | | | biochemistry | | | | | |
| CALCRL | Crohns | categorical | Digestive | 1851 | 375008 | Loss-of-function | 0.127 | 6.96E−05 |
| | Disease | | system disorders | | | | | |
| CALCRL | Crohns | categorical | Digestive | 1851 | 375008 | Loss-of-function | 0.127 | 6.96E−05 |
| | Disease | | system disorders | | | | | |
| EPHB4 | K83 | categorical | Digestive | 2688 | 392153 | Loss-of-function | 0.162 | 2.78E−06 |
| | Diseases of | | system disorders | | | | | |
| | biliary tract | | | | | | | |
| PKN2 | Crohn's | categorical | Digestive | 2266 | 392575 | Loss-of-function | 0.175 | 1.86E−05 |
| | disease - | | system disorders | | | | | |
| | enteritis | | | | | | | |
| CALCRL | Sumatriptan | categorical | Medication | 1073 | 393710 | Loss-of-function | 0.170 | 6.22E−05 |
| | | | history | | | | | |
| SLK | Calceos | categorical | Medication | 218 | 394565 | Loss-of-function | 0.418 | 9.83E−06 |
| | | | history | | | | | |
| EPHB4 | IOP | continuous | NA | 83332 | NA | Loss-of-function | 0.167 | 1.11E−07 |
| | Corneal | | | | | | | |
| | right | | | | | | | |
| MAP3K11 | Corneal | continuous | NA | 83332 | NA | Loss-of-function | 0.207 | 1.55E−74 |
| | hysteresis | | | | | | | |
| MAP3K11 | Corneal | continuous | NA | 83332 | NA | Loss-of-function | 0.184 | 2.85E−58 |
| | resistance | | | | | | | |
| | factor | | | | | | | |
| MAP3K11 | Z47.5 Skin | categorical | Summary | 775 | 394066 | Loss-of-function | 0.219 | 5.37E−05 |
| | of chin | | Operations | | | | | |
| PKN2 | Extraction | categorical | Summary | 770 | 394071 | Loss-of-function | 0.245 | 3.11E−05 |
| | of bone | | Operations | | | | | |
| | marrow | | | | | | | |
| PKN2 | Brachial | categorical | Summary | 189 | 394652 | Loss-of-function | 0.347 | 4.62E−05 |
| | artery | | Operations | | | | | |

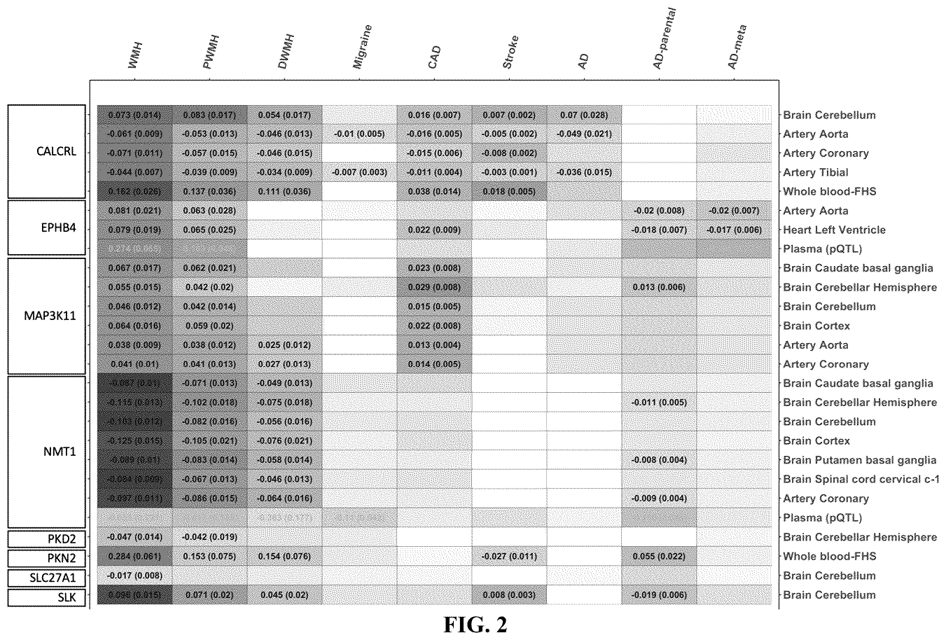

Causal effect estimation using MR for the 11 PCTs identified by INTACT, confirmed the causal relation of 8 PCTs with WMH levels (FIG. 2, Table 10). A consistent effect direction and magnitude were also observed with regional WMH classifications (periventricular WMH, PWMH; deep WMH, DWMH). Notably, changes in the expression levels of CALCRL, NMT1, MAP3K11, and EPHB4 showed a putative causal effect with AD traits in multiple tissue types. The associated AD traits were either clinically defined AD [AD], AD defined by parental status [AD-parental] or meta-analysis of both [AD-meta]). Except for NMT1, the rest showed additional association with related (vascular) endpoints (Stroke; coronary artery disease, CAD; Migraine). The MR analysis suggested lower expression levels of CALCRL and MAP3K11 in the brain, lowering AD risk. Conversely, higher expression levels of NMT1 in multiple tissue types were found to decrease both the risk of AD and the levels of global and regional WMH burden. Additionally, the analysis revealed an inverse relationship for EPHB4, where higher expression levels were associated with a larger WMH burden, but a reduced risk for AD.

TABLE 10
Prioritized PCTs showing significant causal association
(P < 0.05) in the MR analysis with AD and related traits

| PCT-HGNC | Outcome | Beta | StdErr | pvalue | Tissue type | Tissue group | Dataset |
|---|---|---|---|---|---|---|---|
| CALCRL | AD | −0.049 | 0.021 | 2.11E−02 | Artery_Aorta | Vascular | GTEx |
| CALCRL | CAD | −0.016 | 0.005 | 1.34E−03 | Artery_Aorta | Vascular | GTEx |
| CALCRL | DWMH | −0.046 | 0.013 | 3.15E−04 | Artery_Aorta | Vascular | GTEx |
| CALCRL | Migraine | −0.01 | 0.005 | 3.51E−02 | Artery_Aorta | Vascular | GTEx |
| CALCRL | PVWMH | −0.053 | 0.013 | 3.55E−05 | Artery_Aorta | Vascular | GTEx |
| CALCRL | Stroke | −0.005 | 0.002 | 1.06E−02 | Artery_Aorta | Vascular | GTEx |
| CALCRL | WMH | −0.061 | 0.009 | 1.06E−10 | Artery_Aorta | Vascular | GTEx |
| CALCRL | CAD | −0.015 | 0.006 | 9.47E−03 | Artery_Coronary | Vascular | GTEx |
| CALCRL | DWMH | −0.046 | 0.015 | 2.31E−03 | Artery_Coronary | Vascular | GTEx |
| CALCRL | PVWMH | −0.057 | 0.015 | 1.85E−04 | Artery_Coronary | Vascular | GTEx |
| CALCRL | Stroke | −0.008 | 0.002 | 3.29E−04 | Artery_Coronary | Vascular | GTEx |
| CALCRL | WMH | −0.071 | 0.011 | 1.76E−10 | Artery_Coronary | Vascular | GTEx |
| CALCRL | AD | −0.036 | 0.015 | 2.11E−02 | Artery_Tibial | Vascular | GTEx |
| CALCRL | CAD | −0.011 | 0.004 | 1.34E−03 | Artery_Tibial | Vascular | GTEx |
| CALCRL | DWMH | −0.034 | 0.009 | 3.15E−04 | Artery_Tibial | Vascular | GTEx |
| CALCRL | Migraine | −0.007 | 0.003 | 3.51E−02 | Artery_Tibial | Vascular | GTEx |
| CALCRL | PVWMH | −0.039 | 0.009 | 3.55E−05 | Artery_Tibial | Vascular | GTEx |
| CALCRL | Stroke | −0.003 | 0.001 | 1.06E−02 | Artery_Tibial | Vascular | GTEx |
| CALCRL | WMH | −0.044 | 0.007 | 1.06E−10 | Artery_Tibial | Vascular | GTEx |
| CALCRL | AD | 0.07 | 0.028 | 1.29E−02 | Brain_Cerebellum | Neurological | GTEx |
| CALCRL | CAD | 0.016 | 0.007 | 1.18E−02 | Brain_Cerebellum | Neurological | GTEx |
| CALCRL | DWMH | 0.054 | 0.017 | 2.16E−03 | Brain_Cerebellum | Neurological | GTEx |
| CALCRL | PVWMH | 0.083 | 0.017 | 2.03E−06 | Brain_Cerebellum | Neurological | GTEx |
| CALCRL | Stroke | 0.007 | 0.002 | 3.28E−03 | Brain_Cerebellum | Neurological | GTEx |
| CALCRL | WMH | 0.073 | 0.014 | 1.46E−07 | Brain_Cerebellum | Neurological | GTEx |
| CALCRL | CAD | 0.038 | 0.014 | 4.68E−03 | FHS_blood | Vascular | FHS |
| CALCRL | DWMH | 0.111 | 0.036 | 1.84E−03 | FHS_blood | Vascular | FHS |
| CALCRL | PVWMH | 0.137 | 0.036 | 1.39E−04 | FHS_blood | Vascular | FHS |
| CALCRL | Stroke | 0.018 | 0.005 | 3.47E−04 | FHS_blood | Vascular | FHS |
| CALCRL | WMH | 0.162 | 0.026 | 5.88E−10 | FHS_blood | Vascular | FHS |
| EPHB4 | AD-meta | −0.02 | 0.007 | 1.83E−03 | Artery_Aorta | Vascular | GTEx |
| EPHB4 | AD- | −0.02 | 0.008 | 1.35E−02 | Artery_Aorta | Vascular | GTEx |
| | parental | | | | | | |
| EPHB4 | PVWMH | 0.063 | 0.028 | 2.64E−02 | Artery_Aorta | Vascular | GTEx |
| EPHB4 | WMH | 0.081 | 0.021 | 1.17E−04 | Artery_Aorta | Vascular | GTEx |
| EPHB4 | AD-meta | −0.017 | 0.006 | 4.18E−03 | Heart_Left_Ventricle | Vascular | GTEx |
| EPHB4 | AD- | −0.018 | 0.007 | 1.39E−02 | Heart_Left_Ventricle | Vascular | GTEx |
| | parental | | | | | | |
| EPHB4 | CAD | 0.022 | 0.009 | 1.95E−02 | Heart_Left_Ventricle | Vascular | GTEx |
| EPHB4 | PVWMH | 0.065 | 0.025 | 9.32E−03 | Heart_Left_Ventricle | Vascular | GTEx |
| EPHB4 | WMH | 0.079 | 0.019 | 2.81E−05 | Heart_Left_Ventricle | Vascular | GTEx |
| MAP3K11 | CAD | 0.013 | 0.004 | 2.60E−03 | Artery_Aorta | Vascular | GTEx |
| MAP3K11 | DWMH | 0.025 | 0.012 | 3.49E−02 | Artery_Aorta | Vascular | GTEx |
| MAP3K11 | PVWMH | 0.038 | 0.012 | 1.26E−03 | Artery_Aorta | Vascular | GTEx |
| MAP3K11 | WMH | 0.038 | 0.009 | 6.33E−05 | Artery_Aorta | Vascular | GTEx |
| MAP3K11 | CAD | 0.014 | 0.005 | 2.60E−03 | Artery_Coronary | Vascular | GTEx |
| MAP3K11 | DWMH | 0.027 | 0.013 | 3.49E−02 | Artery_Coronary | Vascular | GTEx |
| MAP3K11 | PVWMH | 0.041 | 0.013 | 1.26E−03 | Artery_Coronary | Vascular | GTEx |
| MAP3K11 | WMH | 0.041 | 0.01 | 6.33E−05 | Artery_Coronary | Vascular | GTEx |
| MAP3K11 | CAD | 0.023 | 0.008 | 4.15E−03 | Brain_Caudate_basal_ganglia | Neurological | GTEx |
| MAP3K11 | PVWMH | 0.062 | 0.021 | 3.02E−03 | Brain_Caudate_basal_ganglia | Neurological | GTEx |
| MAP3K11 | WMH | 0.067 | 0.017 | 7.69E−05 | Brain_Caudate_basal_ganglia | Neurological | GTEx |
| MAP3K11 | AD- | 0.013 | 0.006 | 2.41E−02 | Brain_Cerebellar_Hemisphere | Neurological | GTEx |
| | parental | | | | | | |
| MAP3K11 | CAD | 0.029 | 0.008 | 1.58E−04 | Brain_Cerebellar_Hemisphere | Neurological | GTEx |
| MAP3K11 | PVWMH | 0.042 | 0.02 | 3.72E−02 | Brain_Cerebellar_Hemisphere | Neurological | GTEx |
| MAP3K11 | WMH | 0.055 | 0.015 | 2.51E−04 | Brain_Cerebellar_Hemisphere | Neurological | GTEx |
| MAP3K11 | CAD | 0.015 | 0.005 | 4.15E−03 | Brain_Cerebellum | Neurological | GTEx |
| MAP3K11 | PVWMH | 0.042 | 0.014 | 3.02E−03 | Brain_Cerebellum | Neurological | GTEx |
| MAP3K11 | WMH | 0.046 | 0.012 | 7.69E−05 | Brain_Cerebellum | Neurological | GTEx |
| MAP3K11 | CAD | 0.022 | 0.008 | 4.15E−03 | Brain_Cortex | Neurological | GTEx |
| MAP3K11 | PVWMH | 0.059 | 0.02 | 3.02E−03 | Brain_Cortex | Neurological | GTEx |
| MAP3K11 | WMH | 0.064 | 0.016 | 7.69E−05 | Brain_Cortex | Neurological | GTEx |
| NMT1 | AD- | −0.009 | 0.004 | 4.41E−02 | Artery_Coronary | Vascular | GTEx |
| | parental | | | | | | |
| NMT1 | DWMH | −0.064 | 0.016 | 3.97E−05 | Artery_Coronary | Vascular | GTEx |
| NMT1 | PVWMH | −0.086 | 0.015 | 2.36E−08 | Artery_Coronary | Vascular | GTEx |
| NMT1 | WMH | −0.097 | 0.011 | 1.52E−17 | Artery_Coronary | Vascular | GTEx |
| NMT1 | DWMH | −0.049 | 0.013 | 1.74E−04 | Brain_Caudate_basal_ganglia | Neurological | GTEx |
| NMT1 | PVWMH | −0.071 | 0.013 | 6.79E−08 | Brain_Caudate_basal_ganglia | Neurological | GTEx |
| NMT1 | WMH | −0.087 | 0.01 | 5.76E−19 | Brain_Caudate_basal_ganglia | Neurological | GTEx |
| NMT1 | AD- | −0.011 | 0.005 | 4.41E−02 | Brain_Cerebellar_Hemisphere | Neurological | GTEx |
| | parental | | | | | | |
| NMT1 | DWMH | −0.075 | 0.018 | 3.97E−05 | Brain_Cerebellar_Hemisphere | Neurological | GTEx |
| NMT1 | PVWMH | −0.102 | 0.018 | 2.36E−08 | Brain_Cerebellar_Hemisphere | Neurological | GTEx |
| NMT1 | WMH | −0.115 | 0.013 | 1.52E−17 | Brain_Cerebellar_Hemisphere | Neurological | GTEx |
| NMT1 | DWMH | −0.056 | 0.016 | 3.51E−04 | Brain_Cerebellum | Neurological | GTEx |
| NMT1 | PVWMH | −0.082 | 0.016 | 1.59E−07 | Brain_Cerebellum | Neurological | GTEx |
| NMT1 | WMH | −0.103 | 0.012 | 5.76E−19 | Brain_Cerebellum | Neurological | GTEx |
| NMT1 | DWMH | −0.076 | 0.021 | 2.52E−04 | Brain_Cortex | Neurological | GTEx |
| NMT1 | PVWMH | −0.105 | 0.021 | 3.34E−07 | Brain_Cortex | Neurological | GTEx |
| NMT1 | WMH | −0.125 | 0.015 | 1.70E−16 | Brain_Cortex | Neurological | GTEx |
| NMT1 | AD- | −0.008 | 0.004 | 4.20E−02 | Brain_Putamen_basal_ganglia | Neurological | GTEx |
| | parental | | | | | | |
| NMT1 | DWMH | −0.058 | 0.014 | 6.33E−05 | Brain_Putamen_basal_ganglia | Neurological | GTEx |
| NMT1 | PVWMH | −0.083 | 0.014 | 7.49E−09 | Brain_Putamen_basal_ganglia | Neurological | GTEx |
| NMT1 | WMH | −0.089 | 0.01 | 1.52E−17 | Brain_Putamen_basal_ganglia | Neurological | GTEx |
| NMT1 | DWMH | −0.046 | 0.013 | 3.51E−04 | Brain_Spinal_cord_cervical_c-1 | Neurological | GTEx |
| NMT1 | PVWMH | −0.067 | 0.013 | 1.59E−07 | Brain_Spinal_cord_cervical_c-1 | Neurological | GTEx |
| NMT1 | WMH | −0.084 | 0.009 | 5.76E−19 | Brain_Spinal_cord_cervical_c-1 | Neurological | GTEx |
| PKD2 | PVWMH | −0.042 | 0.019 | 2.78E−02 | Brain_Cerebellar_Hemisphere | Neurological | GTEx |
| PKD2 | WMH | −0.047 | 0.014 | 8.58E−04 | Brain_Cerebellar_Hemisphere | Neurological | GTEx |
| PKN2 | AD- | 0.055 | 0.022 | 1.16E−02 | FHS_blood | Vascular | FHS |
| | parental | | | | | | |
| PKN2 | DWMH | 0.154 | 0.076 | 4.16E−02 | FHS_blood | Vascular | FHS |
| PKN2 | PVWMH | 0.153 | 0.075 | 4.19E−02 | FHS_blood | Vascular | FHS |
| PKN2 | Stroke | −0.027 | 0.011 | 1.24E−02 | FHS_blood | Vascular | FHS |
| PKN2 | WMH | 0.284 | 0.061 | 3.38E−06 | FHS_blood | Vascular | FHS |
| SLC27A1 | WMH | −0.017 | 0.008 | 3.03E−02 | Brain_Cerebellum | Neurological | GTEx |
| SLK | AD- | −0.019 | 0.006 | 1.03E−03 | Brain_Cerebellum | Neurological | GTEx |
| | parental | | | | | | |
| SLK | DWMH | 0.045 | 0.02 | 2.30E−02 | Brain_Cerebellum | Neurological | GTEx |
| SLK | PVWMH | 0.071 | 0.02 | 3.49E−04 | Brain_Cerebellum | Neurological | GTEx |
| SLK | Stroke | 0.008 | 0.003 | 4.75E−03 | Brain_Cerebellum | Neurological | GTEx |
| SLK | WMH | 0.096 | 0.015 | 7.19E−11 | Brain_Cerebellum | Neurological | GTEx |

Further validation at the pQTL level provided additional support for the association of NMT1 and EPHB4 with consistent effect direction in plasma with the associated traits, as observed in the eQTL-based analysis (FIG. 2). However, our meta-analysis strategy failed to identify suitable pQTL instruments for validating the causal association of the other genes. The colocalization analysis revealed that a shared pQTL (rs1053733) is regulating the protein levels of NMT1 and trait levels/risk (WMH, PWMH, DWMH, Migraine, AD-parental) (Table 11). On the contrary, different pQTLs that are in weak LD (r2-0.09) with each other regulate the protein levels of EPHB4 and WMH levels (rs314337) and AD risk (rs909152). Moreover, testing the association of protein signatures from post-mortem brain tissues with hippocampal volume confirmed the association of higher levels of EPHB4 proteins with hippocampal atrophy (Table 12).

TABLE 11
Colocalization probability for shared pQTL between
protein levels and trait levels/risk

| | | | SNP Colocalization |
|---|---|---|---|
| PCT-HGNC | Trait | SNP | prob. |
| NMT1 | WMH | rs1053733 | 1.000 |
| NMT1 | PWMH | rs1053733 | 1.000 |
| NMT1 | DWMH | rs1053733 | 1.000 |
| NMT1 | MIGRAINE | rs1053733 | 1.000 |
| NMT1 | AD-Parental | rs1053733 | 1.000 |
| EPHB4 | PWMH | rs314337* | 1.000 |
| EPHB4 | WMH | rs314337 | 1.000 |
| EPHB4 | AD-meta | rs909152 | 1.000 |
| EPHB4 | AD-Parental | rs909152 | 1.000 |

*rs314337 and rs909152 are LD independent based on 1000G EUR (r2 = 0.09)

TABLE 12
Protein level association of prioritized PCTs with
hippocampal volume in FHS - prefrontal cortex

| PCT-HGNC | UniprotID | Protein name | Beta | StdErr | Z score | pvalue | N |
|---|---|---|---|---|---|---|---|
| EPHB4 | P54760 | Ephrin type-B receptor 4 | −0.052 | 0.018 | −2.932 | 3.36E−03 | 956 |
| PKN2 | Q16513 | Serine/threonine-protein | 0.028 | 0.017 | 1.684 | 9.21E−02 | 956 |
| | | kinase N2 | | | | | |
| GALK1 | P51570 | Galactokinase | −0.022 | 0.017 | −1.318 | 1.87E−01 | 956 |
| PKD2 | Q13563 | Polycystin-2: Cytoplasmic | 0.021 | 0.016 | 1.303 | 1.93E−01 | 956 |
| | | domain 4 | | | | | |
| NOS3 | P29474 | Nitric oxide synthase 3 | 0.017 | 0.017 | 0.994 | 3.20E−01 | 956 |
| NMT1 | P30419 | Glycylpeptide N- | −0.016 | 0.017 | −0.947 | 3.44E−01 | 956 |
| | | tetradecanoyltransferase 1 | | | | | |
| ARNT | P27540 | Aryl hydrocarbon receptor | 0.007 | 0.020 | 0.377 | 7.06E−01 | 956 |
| | | nuclear translocator | | | | | |
| MAP3K11 | Q16584 | Mitogen-activated protein | −0.004 | 0.017 | −0.248 | 8.04E−01 | 956 |
| | | kinase kinase kinase 11 | | | | | |

Druggable pathways in WMH: AD association and causal effect of the pathway components. Studying the joint effect of common variants that are proximal to the genes in pathways that can be putatively modulated by pharmacological compounds; showed enrichment for seven clinically actionable pathways for WMH at varying levels of polygenicity (Table 1). The strongest association was observed for the hypoxia pathway, which includes genes that are known to be up-regulated in response to low oxygen levels (P value=1.30×10^{-4, β=25}, P_{T}=. 005). The second strongest association was observed for the thrombin signaling pathway specifically mediated by the Protease Activated Receptor 4 (PAR4) and involved in hemostasis (P value=2.94×10⁻⁴, β=.51, P_{T}=.5). We also found an enrichment of genes involved in the coagulation cascade (P value=4.80×10⁻⁴, β=.15, P_{T}=.05), supporting the association of WMH with the thrombin signaling pathway. Additionally, pathways involved in cellular homeostasis (Downstream effectors of P53 and the RhoA signaling pathway) and extracellular matrix maintenance (genes encoding extracellular matrix and extracellular-matrix associated proteins) were amongst the enriched signals, thereby providing a pharmacological perspective from the genetic burden.

TABLE 1
Pathways enriched in common variations associated with
WMH and potential drug repurposing opportunities.

| | | | | P |
|---|---|---|---|---|
| | No of | | | Threshold |
| Pathway | Genes | Effect | P value | (P_{T}) |
| Hypoxia pathway | 26 | 0.250 | 1.30E−04 | 0.005 |
| PAR4-mediated thrombin | 15 | 0.509 | 2.94E−04 | 0.5 |
| events | | | | |
| P53 downstream effectors | 72 | 0.144 | 3.04E−04 | 0.05 |
| Coagulation pathway | 63 | 0.145 | 4.80E−04 | 0.05 |
| RhoA signaling pathway | 44 | 0.410 | 6.76E−04 | 1 |
| Matrisome pathway | 946 | 0.064 | 8.90E−04 | 0.5 |

P threshold (P_{T}) indicates SNPs below the association threshold in the WMH GWAS, contributing to the pathway enrichment analysis.

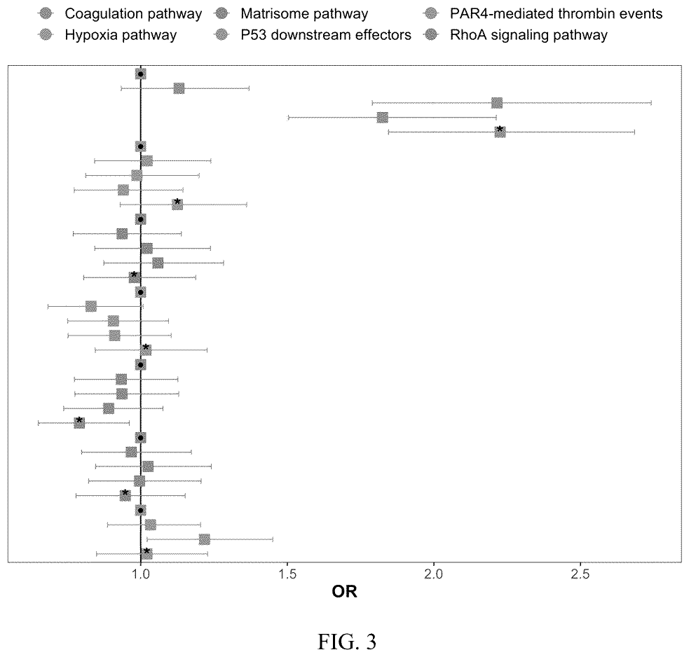

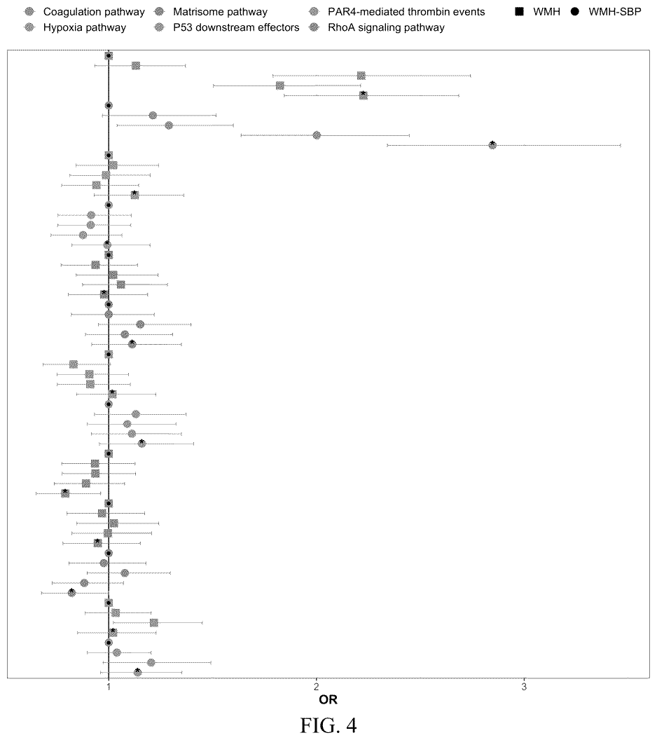

Further, in the UK Biobank cohort, we studied whether individuals with a higher genetic burden for a specific candidate pathway have a higher dementia risk compared to the reference population. From the enriched pathways in WMH (Table 1), we generated pathway-specific PGS in the UK Biobank cohort weighting on the WMH effects. We then tested the association between these WMH-PGS and dementia (AD) status using logistic regression. Our findings showed that individuals with a high genetic burden for coagulation events had an elevated risk for AD (HR: 2.23, CI: 1.85-2.69, P=1.88×10⁻⁴) (FIG. 3). The risk increased further (HR: 2.85, CI: 2.34-3.46, P=2.56×10⁻⁵) when we enhanced the predictive power of the PGS by considering the genetic correlation of WMH with SBP (FIG. 4).

### C. Discussion

In this study, capitalizing on the genetic determinants (eQTLs and pQTLs) reflecting changes in the cellular levels of drug targets across diverse tissue types, we prioritize a list of drug-targets (n=11) with putative causal effect on the preclinical MRI markers of AD and AD risk. We employed statistical fine-mapping techniques to identify genetic instruments specific to each drug target, controlling for confounding and off-target associations that may be influenced by the non-tested drug targets. Changes in the gene expression levels of CALCRL, MAP3K11, and NMT1 appear to be showing consistent effect direction for WMH, AD and multiple AD-related outcomes in two or more tissue types, indicating target-specific putative causal effects. With inhibitory (antagonist) effect as the most prominent drug-gene interaction type between the prioritized drug targets and pharmacological compounds. A phenome-wide scan assessing the loss-of-function of the genes encoding the drug targets, and thus mimicking the inhibitory effect of pharmacological compounds, suggested a risk of digestive complications. We observed a concordance in the effect direction at the protein level, for the association of genetically predicted higher protein levels of NMT1 with lowered WMH burden and reduced AD risk. Further, studying the druggable pathways that can be modulated by pharmacological compounds showed pathways involved in cellular homeostasis and hemostasis to be enriched for larger WMH burden. Interestingly, WMH polygenic scores generated from the enriched pathways showed a higher genetic burden for coagulation events to be associated with a higher risk for AD.

CALCRL, the gene encoding the neuropeptide receptor-calcitonin gene-related peptide type 1 receptor (CGRPr), plays a pivotal role in migraine pathophysiology. The binding of its peptide ligand, calcitonin gene-related peptide (CGRP), has been implicated in vasodilation and inflammation. The CGRPr-CGRP binding mediates the cyclic AMP (CAMP) production⁴⁶ and causes vasodilation of smooth muscle cells, including those in the cerebral vasculature,⁴⁷ either directly or through an endothelial-dependent mechanism.^{48,49} Immunohistochemical studies suggest the localization of CGRPr in the glial⁵⁰ and neuronal cells of the cerebral cortex, cerebellum and hippocampus,⁵¹ potentially contributing to neurogenic inflammation in the central nervous system by releasing pro-inflammatory molecules.⁵² Recent insights connect additional pathological processes of migraine (cortical spreading depression) that are potentiated by CGRPr activation⁵³ with stroke aetiology and poor functional outcomes after stroke.^{54,55} Interestingly, all these processes have been mechanistically linked to both hypoperfusion and blood-brain barrier dysfunction^{56,57}—the pathological hallmarks of white matter hyperintensities (WMH).¹⁴ In this context, our observation of a putative causal association between higher CALCRL expression levels and WMH burden, and genetic liability to stroke and AD, underlines a promising drug repurposing strategy. Small molecules (gepants) and monoclonal antibodies inhibiting the CGRPr activity with demonstrated efficacy for migraine treatment could be studied for targeting the preclinical stages of the AD process. Notably, recent in vivo studies on AD mouse models indicate administering CGRPr antagonists reduces AD pathological features, including amyloid-β levels and tau aggregation, while also enhancing cognitive function by increasing the expression of postsynaptic protein (PSD95).⁵⁸ Although most CGRPr antagonists have a favorable safety profile without treatment-associated adverse events like cardiovascular disease or stroke.⁵⁹⁻⁶¹ Few studies that are reporting adverse effects such as cerebral ischemia,^{62,63} are likely reflective of inherent study limitations, including differences in pharmacokinetics and dose range between animal models and humans,⁶² or a lack of screening for pathogenic mutations contributing to disease onset.⁶³

A common theme arises in the context of other drug targets (MAP3K11, NMT1), underscoring their role in post-translation modifications (PTMs) and maintaining cellular homeostasis and survival. In-depth molecular profiling of post-mortem human brain tissues has offered valuable insights into PTMs and their regulators, linking various PTMs to different stages of AD pathology and showcasing their therapeutic potential in preventing or delaying AD progression.^{64,65} MAP3K11, predominantly expressed in neurons, encodes mixed-lineage kinase-3 (MLK3) and serves as a crucial regulator of the MAPK signaling pathway through PTM-phosphorylation. This process promotes cell death under cellular and metabolic stress conditions. Inhibitors targeting MLK3 and impeding the downstream apoptosis cascade have demonstrated neuroprotective properties in multiple in vivo studies,⁶⁶ and studied in clinical trials for other neurodegenerative disorders. Our observation of lowered MAP3K11 expression levels with reduced genetic liability to AD risk and, in conjunction with other studies suggesting MLK3 modulation in reducing amyloid-β induced toxicity,⁶⁷ place MLK3 inhibitors in a pertinent context with AD. Moreover, the observed lower WMH burden as an effect of lowering the MAP3K11 expression levels supports the effectiveness of targeting AD preclinical stages. The development of pharmacological compounds highly selective to MLK3 with high brain penetrance, crossing the blood-brain barrier is an encouraging prospect.⁶⁸ Indeed, in vivo studies on cerebral ischemia inhibiting post-translational modifications and activation of MLK3 resulted in lowering the inflammatory cytokine levels and a decrease in the cerebral infarct volume in a dose-dependent manner.^{69,70} Finally, the novel association of NMT1 (N-myristoyl transferase-1) with reduced WMH burden and AD risk, highlights the significance of N-myristoylation-a relatively underexplored post-translational modification (PTM) in the AD process. This is particularly noteworthy considering the relevance of N-myristoylation in the endocytosis pathway and its role in regulating amyloid-β peptide levels,^{71,72} presenting a potentially impactful therapeutic target for AD.

MRI-defined vascular brain injuries precede plaque formation by several decades, whose additive effect on amyloid burden could potentiate the development of AD from presymptomatic stages.^{73,74} By studying the druggable pathways in such covert processes, our study highlights the enrichment of mechanisms governing hemostasis, blood vessel integrity, endothelial function and angiogenesis. Perturbations to such physiological processes have been shown to play a central role in the pathophysiology of nonamyloid cerebral small vessel disease.^{75,76} As a novel finding, we observed individuals with a higher genetic burden for coagulation, showing more than doubled risk for the Alzheimer's type of dementia, highlighting potential vascular mechanisms underpinning neuronal dysfunction and neurodegenerative processes. Notably, this is consistent with the observations in AD mouse models suggesting the role of vascular damage and specific coagulation proteins in neuroinflammation and cognitive decline.^{77,78} Briefly, at the sites of vascular damage, fibrinogen deposition has been shown to induce a pathogenic microglia response promoting the loss of dendritic spines and cognitive impairment. Moreover, our observation of the association of coagulation processes with AD only in the presence of WMH effects provides additional support to the fibrinogen-mediated brain pathology to be region-specific relating to the degree of blood-brain barrier dysfunction.⁷⁷ The binding of fibrinogen with amyloid-beta is discussed as one possible mechanism for blocking fibrin degradation and promoting fibrin-induced microglia activation and neuroinflammation.⁷⁹ Finally, in the context of drug-repurposing strategies, it becomes crucial to investigate the potential causal links between these coagulation cascade components and amyloid-beta pathology. As such exploration will facilitate identifying the substrates that interact specifically in an amyloid-beta-rich environment and developing targeting strategies while maintaining the essential hemostatic properties.

Our study boasts notable strengths. Firstly, it integrates cellular phenotypes of drug targets from large population-based studies, using advanced methods to define genetic instruments. Secondly, employing MR and complementary analyses, we prioritize drug targets with consistent effects on WMH traits, AD, and related endpoints, offering insights for target-specific drug trials. Lastly, our pathway-based polygenic score analyses, conducted in a non-overlapping sample set, underscore the crucial vascular components linking vascular pathology and neurodegeneration. Moreover, the analytical scheme developed could be extended to explore other brain imaging features, including perivascular spaces, that are integral to the glymphatic system and directly implicated in amyloid-beta clearance.⁸²

In summary, our study provides a tractable roadmap for drug-target prioritization within the druggable genome by triangulating evidence from population genomic, transcriptomic and proteomic data. Multiple lines of evidence suggest certain drug targets to have a causative role in WMH burden and AD risk. This goes beyond biomarker functions, emphasizing drug-repurposing opportunities and supporting rationale for clinical trials. Additionally, the shared gene function among prioritized targets underscores the importance of post-translational modification in the AD disease process. Lastly, from a genetic epidemiology standpoint, our study provides novel insights into the connection between vascular brain injury, the coagulation cascade, and AD risk, highlighting specific components with potential causal roles.

The examples as well as the figures are included to demonstrate preferred embodiments of the invention. It should be appreciated by those of skill in the art that the techniques disclosed herein represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### III. Treatment of Subjects

The determination of an appropriate treatment regimen (e.g., dosage, frequency of administration, systemic vs. local, etc.) for mitigating, preventing, delaying, or treating dementia associated with white matter hyperintensity (WMH) burden, cerebrovascular disease, or neurodegenerative processes can be performed using the information provided herein.

The therapeutic agents described herein include drugs that modulate the putative causal targets (PCTs) identified, such as inhibitors of CALCRL (encoding the calcitonin gene-related peptide receptor, CGRPr), inhibitors of MAP3K11 (encoding mixed-lineage kinase-3, MLK3), modulators of NMT1 (N-myristoyltransferase-1), and agents targeting pathways enriched for WMH, including anticoagulants or inhibitors of the coagulation cascade (e.g., thrombin signaling or PAR4-mediated pathways).

In certain embodiments, the therapeutic agent is a CGRPr antagonist. Exemplary CGRPr antagonists include small-molecule gepants, such as: Rimegepant, Ubrogepant, atogepant, zavegepant, and monoclonal antibodies targeting CGRP or the CGRP receptor, such as: erenumab, fremanezumab, galcanezumab, eptinezumab. These agents, originally approved for migraine prevention and/or acute treatment, are repurposed herein for reducing WMH burden and mitigating dementia risk, particularly Alzheimer's disease.

In other embodiments, the therapeutic agent is an inhibitor of mixed-lineage kinase-3 (MLK3), which may include small-molecule inhibitors currently in development or identified as modulating MAP3K11 activity.

In further embodiments, the therapeutic agent is a modulator (e.g., inhibitor) of NMT1, including compounds such as PCLX-001 (zelenirstat) or other N-myristoyltransferase inhibitors in preclinical or clinical development.

In additional embodiments, the therapeutic agent is an anticoagulant or an inhibitor targeting the coagulation cascade, such as agents that modulate thrombin signaling, PAR4, or related hemostasis pathways.

For administration, the components described herein will be formulated in a unit dosage form (solution, suspension, emulsion, tablet, capsule, orally disintegrating tablet, nasal spray, injection, etc.) in association with a pharmaceutically acceptable carrier. Such vehicles are usually nontoxic and non-therapeutic. Examples of such vehicles include: water, saline, Ringer's solution, dextrose solution, Hank's solution, etc.

Non-aqueous vehicles such as fixed oils and ethyl oleate may also be used. A preferred vehicle is 5% (w/w) human albumin in saline. The vehicle may contain minor amounts of additives, such as substances that enhance isotonicity and chemical stability, e.g., buffers and preservatives.

The therapeutic compositions described herein, as well as their biological equivalents or pharmaceutically acceptable salts thereof, can be administered independently or in combination with other agents by any suitable route. Examples of routes of administration include: oral, intravenous, subcutaneous, intramuscular, intranasal, intraperitoneal, intrathecal, and/or topical. The routes of administration described herein are merely examples and in no way limiting.

The dose of the therapeutic compositions administered to a subject, particularly a human, in accordance with embodiments of the invention, should be sufficient to result in a desired response, such as reduction in WMH burden, stabilization or improvement in cognitive function, or delay in dementia onset, over a reasonable time frame. The dosage depends upon a variety of factors, including: the strength of the particular therapeutic composition employed; the age, species, condition, or disease state of the subject; the body weight of the subject; and/or the timing of administration relative to disease progression. Dose and dosage regimen will depend mainly on the type and extent of vascular or neurodegenerative damage, the subject's history, and the type of therapeutic composition being administered. The size of the dose will be determined by the route, timing, and frequency of administration as well as the existence, nature, and extent of any adverse side effects and the desired physiological effect. Various conditions or disease states, in particular chronic conditions, may require prolonged treatment involving multiple administrations.

The amount of the therapeutic composition must be effective to achieve an enhanced therapeutic index for dementia prevention or treatment. If multiple doses are employed, the frequency of administration will depend on the type of subject and pharmacokinetic properties of the agent. One skilled in the art can ascertain upon routine experimentation the appropriate route, dosage, and frequency of administration in a given subject to achieve the most effective outcome. Suitable doses and dosage regimens can be determined by conventionally known range-finding techniques. Generally, treatment is initiated with smaller dosages, which are less than the optimal dose of the compound. Thereafter, the dosage is increased by small increments until the optimal effect under the circumstances is obtained.

In certain embodiments, subjects are selected for treatment based on elevated WMH burden as assessed by brain imaging (e.g., MRI), presence of vascular risk factors, genetic predisposition (e.g., polygenic scores for WMH-enriched pathways), or early signs of cognitive impairment. Combination therapies, such as a CGRPr antagonist with an anticoagulant or other neuroprotective agents, are also contemplated.

The terms “treating” or “treatment” refer to any success or indicia of success in the attenuation or amelioration of an injury, pathology, or condition, including any objective or subjective parameter such as abatement, remission, diminishing of symptoms, making the injury, pathology, or condition more tolerable to the patient, slowing in the rate of degeneration or decline, improving physical or mental well-being, or prolonging survival. Treatment or amelioration of symptoms can be based on objective or subjective parameters, including results of a physical examination, neurological examination, brain imaging, or cognitive assessments.

An “effective amount” or “therapeutically effective amount” refers to an amount of a therapeutic agent sufficient to achieve a particular biological or therapeutic result, such as reduction in WMH progression, mitigation of vascular contributions to dementia, or delay in Alzheimer's disease onset. A therapeutically effective amount may vary according to factors such as the disease state, age, sex, and weight of the individual, and the ability of the agent to elicit a desired response.

### REFERENCES

1. Prince et al., Alzheimer's Research & Therapy 8 (2016).
2. Dubois et al., Lancet Neurol 20, 484-496 (2021).
3. Tai et al., The Lancet Healthy Longevity 3, e428-e436 (2022).
4. Song et al., Alzheimer's & Dementia 17, 1914-1922 (2021).
5. Savva and Stephan, A systematic review. Vol. 41 (Stroke, 2010).
6 Nation et al., Nature Medicine 25, 270-276 (2019).
7. van de Haar et al., Radiology 281, 527-535 (2016).
8. Malik et al., Alzheimer's & Dementia 17, 1422-1431 (2021).
9. Sargurupremraj et al., Nature Communications 11 (2020).
10. Power et al., Epidemiology 22, 646-59 (2011).
11. Van Dalen et al., JAMA Internal Medicine 182, 142 (2022).
12. Parati et al., Nat Rev Cardiol 10, 143-55 (2013).
13. Prins and Scheltens, Nature Reviews Neurology 11, 157-165 (2015).
14. Debette and Markus, BMJ 341, c3666-c3666 (2010).
15. Wardlaw et al., Vol. 12 822-838 (Lancet Neurol, 2013).
16. Sargurupremraj et al., Complexities of cerebral small vessel disease, blood pressure, and dementia relationship: new insights from genetics. (Cold Spring Harbor Laboratory, 2023).
17. Daghlas and Gill, Cambridge Prisms: Precision Medicine 1, 1-36 (2023).
18. Georgakis et al., Neurology 95, e353-e361 (2020).
19. Bordes et al., Nature Reviews Neurology (2022).
20. Armstrong et al., Stroke 51, 2111-2121 (2020).
21. Bellenguez et al., Nature Genetics 44, 328-333 (2012).
22 Boyle and Pritchard, Cell 169, 1177-1186 (2017).
23. Anderson and Williams, Diabetologia 66, 1156-1158 (2023).
24. Finan et al., Science Translational Medicine 9, eaag1166 (2017).
25. Okamoto et al., Am J Hum Genet 110, 44-57 (2023).
26. Lonsdale et al., Nature Genetics 45, 580-585 (2013).
27. Pietzner et al., Science, eabj1541 (2021).
28. Ferkingstad et al., Nature Genetics 53, 1712-1721 (2021).
29. Sun et al., Nature 558, 73-79 (2018).
30. Wen et al., The American Journal of Human Genetics 98, 1114-1129 (2016).
31. Zhang et al., Genome Biology 21 (2020).
32. Hukku et al., Am J Hum Genet 109, 825-837 (2022).
33. Hukku et al., The American Journal of Human Genetics 108, 25-35 (2021).
34. Bowden et al., International Journal of Epidemiology 47, 1264-1278 (2018).
35. Bowden et al., International Journal of Epidemiology 44, 512-525 (2015).
36. Swerdlow et al., International Journal of Epidemiology 45, 1600-1616 (2016).
37. Joehanes et al., Genome Biology 18 (2017).
38. Consortium, Science 369, 1318-1330 (2020).
39. Yang et al., Nat Neurosci 24, 1302-1312 (2021).
40. Wang et al., PLOS Genetics 17, e1009575 (2021).
41. Zhao et al., The Annals of Statistics 48, 1742-1769, 28 (2020).
42. Lawlor et al., International Journal of Epidemiology, dyw314 (2017).
43. Giambartolomei et al., Bioinformatics 34, 2538-2545 (2018).
44. Reay et al., Scientific Reports 10 (2020).
45. Choi and O'Reilly, Gigascience 8 (2019).
46. McLatchie et al., Nature 393, 333-9 (1998).
47. Oliver et al., J Cereb Blood Flow Metab 22, 620-9 (2002).
48. Kumar et al., Frontiers in Physiology 10 (2019).
49. Zhang et al., Circulation Research 133, 902-923 (2023).
50. Bower et al., Am J Physiol Regul Integr Comp Physiol 310, R788-93 (2016).
51. Warfvinge and Edvinsson, Cephalalgia 39, 342-353 (2019).
52. Raddant and Russo, Expert Reviews in Molecular Medicine 13 (2011).
53. Tozzi et al., PNAS 109, 18985-18990 (2012).
54. Osteraas and Dafer, Current Neurology and Neuroscience Reports 23, 301-325 (2023).
55. Wang et al., European Stroke Journal 8, 517-521 (2023).
56. Mason and Russo, Frontiers in Cellular Neuroscience 12 (2018).
57. Woods et al., N Engl J Med 331, 1689-92 (1994).
58. Na et al., Neuropharmacology 168, 108017 (2020).
59. Hutchinson et al., Cephalalgia 41, 979-990 (2021).
60. Gomez et al., Stroke 52, AP643-AP643 (2021).
61. Giamberardino et al., Journal of Pain Research Volume 10, 2751-2760 (2017).
62. Aradi et al., J Stroke Cerebrovasc Dis 28, 104286 (2019).
63. Mulder et al., Ann Neurol 88, 771-784 (2020).
64. Kelley et al., Biochim Biophys Acta Mol Basis Dis 1865, 2040-2047 (2019).
65. Ercan-Herbst et al., Acta Neuropathologica Communications 7 (2019).
66. Moreno et al., Eur J Med Chem 257, 115511 (2023).
67. Xu et al., J Neurosci Res 87, 918-27 (2009).
68. Kline et al., Exp Neurol 318, 157-164 (2019).
69. Hu et al., Journal of Biological Chemistry 287, 2364-2377 (2012).
70. Yu et al., Frontiers in Pharmacology 11 (2020).
71. Laux et al., J Cell Biol 149, 1455-72 (2000).
72. Su et al., Neurosci Bull 26, 338-44 (2010).
73. Vemuri et al., Brain 138, 761-771 (2015).
74. Debette et al., JAMA Neurology 76, 81 (2019).
75. Joutel et al., J Cereb Blood Flow Metab 36, 143-157 (2016).
76. Kang et al., Stroke 53, 1570-1579 (2022).
77. Merlini et al., Neuron 101, 1099-1108.e6 (2019).
78. Cortes-Canteli et al., Journal of Alzheimer's Disease 32, 599-608 (2012).
79. Ahn et al., PNAS 107, 21812-21817 (2010).
80. Cortes-Canteli et al., Neuron 66, 695-709 (2010).
81. Hanas et al., Axis. Brain Sci 11 (2021).
82. Iliff et al., Science Translational Medicine 4, 147ra111-147ral (2012).
83. Yamamoto et al., Nature Communications 13 (2022).
84. Cruchaga, Alzheimer's & Dementia 18 (2022)

1. A method for identifying a drug for the treatment of a neurodegenerative disease comprising:
selecting genetic variants as proxies for effects of a drug target;
assessing subjects having a neurodegenerative condition relative to the selected genetic variants;
identifying a drug target having a causal relationship with the neurodegenerative disease; and
identifying a drug that modulates the drug target as a therapeutic for the neurodegenerative disease.

2. The method of claim 1, wherein the genetic variants include single-nucleotide polymorphisms (SNP).

3. The method of claim 1, wherein the neurodegenerative disease is Alzheimer's disease or dementia.

4. The method of claim 1, wherein the drug target is selected from CALCRL, MAP3K11, NMT1, EPHB4, GALK1, PKD2, and combinations thereof.

5. The method of claim 1, wherein the drug is an inhibitor of CALCRL (CGRPr), an inhibitor of MAP3K11 (MLK3), an inhibitor of NMT1, or an anticoagulant.

6. A method for treating dementia comprising: administering a drug identified in claim 1 to a subject having, suspected of having, or at risk of developing dementia.

7. The method of claim 6, wherein the subject at risk of developing dementia has an increase in white matter hyperintensity burden.

8. The method of claim 6, wherein the subject at risk of developing dementia has vascular risk factors implicated in Alzheimer's disease pathogenesis.

9. The method of claim 6, wherein the drug identified is an inhibitor of CGRPr activity, inhibitor of mixed-lineage kinase-3 (MLK3), or an anticoagulant.

10. A method of treating or preventing dementia in a subject comprising administering to the subject an effective amount of a CGRPr antagonist.

11. The method of claim 10, wherein the CGRPr antagonist is a gepant or a monoclonal antibody targeting CGRP or CGRPr.

12. The method of claim 11, wherein the monoclonal antibody is erenumab, fremanezumab, galcanezumab, or eptinezumab.

13. A method of treating or preventing dementia in a subject comprising administering to the subject an effective amount of an inhibitor of mixed-lineage kinase 3 (MLK3).

14. A method of treating or preventing dementia in a subject comprising administering to the subject an effective amount of an anticoagulant or an inhibitor of the coagulation cascade.

15. The method of any one of claims 10 to 14, wherein the subject has elevated white matter hyperintensity (WMH) burden as determined by brain imaging.

16. The method of any one of claims 10 to 14, wherein the subject is at risk of developing dementia due to vascular risk factors.

17. A pharmaceutical composition comprising a CGRPr antagonist and a pharmaceutically acceptable carrier for use in treating or preventing dementia.

18. Use of a CGRPr antagonist in the manufacture of a medicament for treating or preventing dementia.
